(19) United States
(12) Patent Application Publication
(10) Pub. No.: US 2004/0131702 A1
(43) Pub. Date: Jul. 8, 2004

(54) Use of selenite or preparation containing selenite for treating wounds

(76) Inventors: Jaroslaw Lampe, Vienna (AT); Ursula Mahner-Ehrig, Vienna (AT); Peter Kossler, Mariapfarr (AT); Norbert Fuchs, Mariapfarr (AT)

Correspondence Address: FULBRIGHT & JAWORSKI L.L.P., 600 CONGRESS AVE., SUITE 2400, AUSTIN, TX, 78701, US

(21) Appl. No.: 10/467,736

(22) PCT Filed: Feb. 1, 2002
(86) PCT No.: PCT/AT02/00040

(30) Foreign Application Priority Data
Feb. 13, 2001 (AT) A 215/2001

(51) Int. Cl. A61K 33/04; A61K 7/16

(52) U.S. Cl. 424/702; 424/49

(57) ABSTRACT

The use of preparations containing selenite or selenate, mixed with a pharmaceutically acceptable or food compatible acid, for the preparation of an agent for the treatment of wounds is described.

The invention relates to the application of preparations containing selenite or selenate having low standard redox potential.

On a biochemical level, all metabolic processes in organic living beings (plants, animals, humans) in the sense of growth, differentiating and energy processes are an interaction of reductive and oxidative processes. Basically, these “redox processes” are the result of the transfer of electrons of biochemical reduction equivalents, such as e.g. NADH+H⁺ (electron donator) to atmospheric, molecular oxygen as an oxidizing agent (electron acceptor). Oxidation of our nutrients (fats, carbohydrates, proteins, oxygen) serves the steady maintenance and development of our biological structures.

On the other hand, our very cellular and subcellular structures, the tissues and organs made therefrom, and, basically, any organic individual in its entirety consists of those very structures (nutrients) which have to be constantly supplied from outside to maintain and develop the living organism, which have to be oxidized to gain energy, and which, however, at the same time also have to serve the maintenance of our functional, anatomic and histological structures. Thus, these biological structures are basically just as oxidizable as those nutrients which have to be oxidized to maintain our energy of life. In order to prevent biological structures from “autooxidation” the living organism makes use of endogenous and exogenous “antioxidants”. Such endogenous antioxidants are, among others, enzymes and enzyme systems, such as superoxide dismutase, katalases, peroxidases, cholesterol and reduced glutathione, while exogenous antioxidants are e.g. vitamin A, β-carotene, vitamin E, vitamin C or selenium.

The measure of “antioxidative capacity”, i.e. the readiness to transfer electrons to other atoms and molecules, is expressed quantitatively in the so-called “reduction potential” (standard redox potential). Table 1 below gives an outline of the standard redox potentials of some endogenous and exogenous antioxidants of living organisms:

TABLE 1
Standard redox potentials of some nutrients

| E₀ (Volt) | System |
|---|---|
| +0.82 | O₂/H₂O |
| +0.366 (basic environment) | selenite |
| +0.300 | tocopherol (vitamin E) |
| +0.100 | ubiquinone (coenzyme Q₁₀) |
| +0.08 | ascorbic acid |
| +0 (+0.16 to −0.02 V) | flavonoids |
| −0.12 | riboflavin (vitamin B₂) |
| −0.22 | cystine/cysteine |
| −0.23 | G SH/GSSG |
| −0.29 | thioctic acid (α-lipoic |
| | acid) |
| −0.32 | NADH + H⁺/NAD |
| −0.740 (acid environment) | selenite |

Antioxidants are atoms and molecules (for the human organism primarily nutrient molecules and enzyme complexes) which are faster to react with metabolic radicals than biological structures. Thus, they protect our cell, gene and connective tissue structures by scavenging initiators of metabolism (radicals, peroxides) before they attack e.g. unsaturated fatty acids of our biomembranes or sulfur containing components of vital structure or enzyme proteins. As the above table shows, certain elements, such as e.g. selenium, change their standard redox potential by a change in the pH environment wherein these compounds are dissolved.

Selenium is an essential trace element for higher animals and humans. It has a protective function for proteins against oxidation, e.g. effected by glutathione peroxidase, which contains the amino acid selenocysteine in its active center. Selenium deficiency is said to be associated with rheumatism and grey cataract, and Keshan's disease, which is common in some regions in China, is said to be a selenium deficiency disease. Selenites are able to increase the effect of vitamin E and are responsible for the clearance of mercury and cadmium. Selenium has also been postulated to have a protective effect against carcinogens.

On the other hand, higher concentrations of selenium have a toxic effect. The reason why it is toxic is said to be that selenium can replace sulfur in proteins. As a rule, selenium is discharged as selenate via the kidneys and the intestines. The human body will fall ill if the daily food contains more than 1 μg of selenium/g (but at least 0.02 μg of selenium/g are essential to prevent deficiency symptoms). Altogether, the human body contains about 10 to 15 mg of selenium.

Animals also exhibit symptoms of poisoning if their food contains more than 5 to 10 μg of selenium/g, e.g. growth impairment, loss of hair, softening of horns and hoofs, loss of feathers in birds. However, selenium is also necessary to raise animals, such as chicken, turkey and pigs, and to prevent specific diseases in farm animals, particularly sheep. Therefore, sodium selenite and sodium selenate are necessary as additives in mixed feeds or pasture fertilizers, because feeds of animal and plant origin often do not contain sufficient amounts of selenium naturally or said element is not released in sufficient amounts.

The U.S. Pat. No. 4,668,515 describes a drink containing selenium, which is mixed with citric acid and ascorbic acid, and the solution should have a pH of 2.75 or higher. According to this document, the primary purpose is to achieve a pH of higher than 2.75 by adding citric and ascorbic acid, because—as is assumed in this document—otherwise sodium selenite will be inactivated by acids. This agent that ascorbic acid etc. is added to keep the pH stable.

Further, U.S. Pat. No. 4,668,515 only describes the systemic use of these preparations to stay healthy and to prevent the spontaneous formation of tumors of mammary glands.

Such systemic administration (e.g. oral or i.v.) will cause this composition to be diluted by body fluids (e.g. in the intestines or in the blood), resulting in the neutralization of the antioxidative potential, e.g. due to a change in the pH environment.

The DE 44 37 403 A1 describes antioxidants containing, among others, selenium in organic compounds, but not as selenite or selenate. The subject matter of this document is based on the combination of various antioxidative substances which are presumed to result in a significant synergistic effect.

Moreover, the combination of antioxidants according to DE 44 37 403 A1 is also only provided for internal use, which will again effect dilution.

Novel, improved preparations containing selenium having increased antioxidative potential are described in the Austrian application A 978/2000, which is incorporated in the present application by reference. Such preparations have been proposed for use in improved food and feed technology and pharmacology, and they have been described to be especially effective with respect to preventing peroxidative, radical-induced and viral diseases.

The object of the present invention is to find and develop novel pharmaceutical and cosmetic applications for such preparations containing selenium.

This object is realized according to the present invention by using preparations containing selenite or selenate, mixed with a pharmaceutically acceptable or food compatible acid, for the preparation of an agent for the treatment of wounds.

As mentioned above, such novel preparations containing selenium having increased antioxidative potential are described in the Austrian application A 978/2000.

It has been found that the reduction of the pH value of solutions containing selenium, particularly sodium selenite or selenate solutions, results in a highly increased antioxidative potential compared to a non-pH reduced solution. Such selenium preparations also exhibit surprising therapeutic effects, particularly in radical or peroxide induced diseases.

A wide variety of acids may be used as acidifiers for selenium preparations. For pharmaceutical or cosmetic applications, however, preferably those acidifiers are used which are harmless for animals or humans, such as e.g. citric acid, acetic acid, malic acid, carbonic acid, various fruit acids and mixtures thereof.

Preferably, ascorbic acid is not used as an acidifier, because in certain variants it may cause undesirable interferences with the selenium salts.

Several pathological processes, such as e.g. inflammatory processes, have proven to continue due to an overwhelming number of oxidative processes on the very biochemical level, which can be seen in the increased formation of radicals and peroxides. In particular, wounds develop necroses, i.e. cell disintegration, due to the special pathohistological situation, resulting in an increase in free radicals and peroxides, particularly if necrotic conditions prevail. Due to local ischemia, these radicals and peroxides can not be neutralized via the blood. This leads to a vicious circle on the pathophysiological level: Cell wall damage due to lipid peroxidation results in early cell death; death of the cell results in edema; edema causes compression of arterioles; compression results in impairments of microcirculation, which, in turn, result in local hypoxemia, cell death and release of radicals, which again leads to lipid peroxidation of cell walls of other cells. This vicious circle finally leads to forms of wound complications which resist therapy.

For instance, in the field of dentistry, the healing process of a wound after extraction may be impaired, which is characterized by therapy resistant, severe pain and all generally known conditions resulting therefrom. This phenomenon is called “dolor post extractionem”. Some experiences and publications in the field of dentistry already hint at positive experiences with the use of antioxidative vitamins (e.g. vitamin E or vitamin C) by parenteral or oral administration of these antioxidants at the moment the wound is caused in a series of tooth extractions. Patients thus treated develop i.a. significantly less pain than patient collectives without antioxidant therapy.

The preparations to be used according to the present invention, comprising a pharmaceutically administrable or food compatible form of selenium and a pharmaceutically acceptable or food compatible acid, have a higher antioxidative potential than conventional selenium preparations.

While, for instance, selenite in a basic environment has a standard redox potential of +0.366 Volt, the redox potential of selenite in acid solutions changes to −0.740 Volt. According to the present invention, acidifiers for the solution containing selenium are particularly those acidifiers which are harmless for humans or animals, such as e.g. citric acid, acetic acid, malic acid, carbonic acid, various fruit acids, inorganic acids and mixtures thereof. Preferably, acidification of the agent containing selenium results in a pH of less than 7.0, preferably less than 5.0, particularly less than 4.0. Particularly preferred agent according to the present invention have a pH of between 6.0 and 2.0, particularly between 3.0 and 2.5.

In the preparation according to the present invention selenium is preferably provided in the form of sodium selenite or sodium selenate.

Generally, a wound is understood to be an interruption in the continuity of body tissues, with or without loss of substance, caused by mechanical injury or cellular damage due to physical causes. Several forms may be distinguished, such as e.g. mechanical wounds, which are most frequently caused by an outside impact, particularly cuts and stab wounds (sharp cutting and sharp pointed, respectively), wounds resulting from contusion, laceration, broken skin and abrasion (blunt), wounds resulting from scratching and biting (combined sharp-blunt) and gunshot wounds; thermal wounds caused by the action of heat (burn) or cold (frostbite); chemical wounds, particularly caused by burns with acids and bases; and wounds caused by irradiation, i.e. the action of actinic (ultra violet) and ionizing radiation.

The treatment according to the present invention has proven particularly helpful for mechanical wounds, particularly such mechanical wounds as are caused in medical and surgical interventions, respectively (see dentistry).

As mentioned above, a particular field of application of the present invention is the field of dentistry. Thus, according to the present invention preferably wounds in the dental region are treated, particularly in connection with tooth extractions or tooth implants. In particular, “dolor post extractionem” has proven to be especially successfully treatable with the methods according to the present invention. Thus, parodontal diseases, which are e.g. associated with small wounds, may also be treated according to the present invention. The treatment of papillomatoses, particularly those associated with wounds, is also included in the present invention.

The vicious circle mentioned above has proven to be breakable according to the present invention, and thus the necrotic processes, the impairments of microcirculation, the local hypoxemia, and the cellular damage due to lipid peroxidation may be treated with the preparations according to the present invention. Therefore, the present invention also relates to the treatment of such conditions.

The preparations according to the present invention may not only be provided in the form of aqueous solutions, but may be provided in any pharmaceutically appropriate form. Preferred forms are gels or emulsions, which have proven highly useful particularly for pharmaceutical application because they allow local topical application (also sustained release). Therefore, the preparation according to the present invention may also be provided as an agent to treat wounds, comprising the preparation containing selenium according to the present invention and a pharmaceutically acceptable carrier, particularly in the form of a combination preparation, e.g. as an agent to treat wounds having a solid surface impregnated, mixed, treated etc. with the selenium preparations according to the present invention, e.g. as a plaster, gel, wound implant, etc. Preferably, the agent according to the present invention is prepared for buccal or topical administration.

In the dental region the application according to the present invention as e.g. gels in the form of toothpastes, mouthwashes or implants is particularly preferred.

The invention will be explained in greater detail using the following examples to which it is not restricted, of course.

### EXAMPLES

### Example 1

Dolor Post Extractionem

53 Patients suffering from “dolor post extractionem” were treated and observed during the study. The objectives of the series of observations were the reduction of pain and the improvement in wound healing.

Said 53 patients, 30 women and 23 men (see tables 2 to 4) suffering from the symptoms of “dolor post extractionem” were treated with a preparation of the following composition:

| | |
|---|---|
| sodium selenite pentahydrate | 0.11 g |
| maltodextrin | 0.5 g |
| lemon aroma | 0.1 g |
| citric acid | 0.5 g |
| food coloring | 0.01 g |
| potassium sorbate | 0.1 g |
| sodium benzoate | 0.05 g |
| aqua dest. | 99.29 g |

TABLE 2

| | | | |
|---|---|---|---|
| n = 53 | Sex: | f 30 | 56.6% |
| | | m 23 | 43.4% |

TABLE 3

| | | | | |
|---|---|---|---|---|
| n = 53 | Age: | under 30a | 13 | 24.5% |
| | | 31 to 50a | 31 | 58.5% |
| | | 51a up | 9 | 17.0% |

Method:

After the wound was cleaned using 0.9% sodium chloride solution, the filling, which was impregnated with the solution mentioned above, was put in.

Most of the patients were between 20 and 35 years of age (table 3).

The localizations were as follows:

Maxilla: 34.5% (19 patients)

Mandible: 65.5% (38 patients)

In 30% of the patients (i.e. in 16 cases) diseases such as diabetes mellitus and/or arterial hypertonia were found in the course of the general examination.

In 53% of the patients (28 cases) analgesics and antibiotics had been used up to the beginning of the selenite treatment.

The number of treatments until the patient was pain-free was used in order to assess the effect:

TABLE 4

| Number of | Patients | |
|---|---|---|
| treatments | n = 53 | % |
| 1 | 26 | 49 |
| 2 | 16 | 30 |
| 3 | 9 | 17 |
| 4 | 1 | 2 |
| 7 | 1 | 2 |

Result: 49% of the patients (26 cases) were pain-free after one treatment, 30% of the patients (16 cases) were pain-free after two treatments. So it may be concluded that 79% of all patients (42 cases) were pain-free after one or two treatments.

Thus, the highly surprising results of the present observations of the application of the preparations to be used according to the present invention have shown that the application of antioxidants having a strong (i.e. low) standard redox potential is highly effective in wounds, particularly in inflammatory and/or non-inflammatory, painful processes in the dental region.

### EXAMPLE 2

Treatment of Parodontal Diseases

Parodontal diseases are among the most widely spread health problems. The composition according to example 1 was therefore used as a spray solution in the following 9 patients:

6 patients suffering from stomatitis

3 patients suffering from aphtae

For about 1 to 2 weeks, the patients were administered 1 or 2 spray doses 3 times a day. After the end of treatment, at least a significant improvement of the clinical picture was observed in all 9 cases.

TABLE 3

| | | Year | | | |
|---|---|---|---|---|---|
| Patient | | of | | Duration of | Success of |
| No. | Sex | birth | Diagnosis | therapy | therapy |
| 1 | male | 1974 | gingivostomatitis | 9 days | complete |
| | | | | | regression; |
| | | | | | visible |
| | | | | | improvement |
| 2 | male | 1988 | gingivostomatitis | 13 days | visible |
| | | | | | improvement |
| 3 | female | 1959 | massive | 7 days | complete |
| | | | gingivostomatitis | | regression; |
| | | | | | visible |
| | | | | | improvement |
| 4 | male | 1978 | gingivostomatitis | | significant |
| | | | | | improvement |
| 5 | female | 1942 | gingivostomatitis | 4 days | significant |
| | | | | | improvement |
| 6 | female | 1932 | stomatitis, | 6 days | significant |
| | | | Candida infection | | improvement |
| 7 | male | 1968 | aphtae | 6 days | significant |
| | | | | | improvement |
| 8 | female | 1945 | aphtae | 3 days | significant |
| | | | | | improvement |
| 9 | male | 1972 | aphtae | 4 days | significant |
| | | | | | improvement |

### EXAMPLE 3

Treatment of Papillomatoses

Papillomatoses are cauliflower-like excrescences caused by the so-called Papilloma virus. Histologically, papillomas are usually benign tumors of the surface epithelium localized in the oral mucosa, the urinary tract, at the thighs and lower legs and in the anal and genital mucosa. If a papilloma persists for a long time, it may develop from a Papilloma virus induced benign tumor to carcinoma.

3 female patients (aged 6, 39, 53) had perianal papilloma warts (verrucae) and were treated with a preparation of the following composition:

| | |
|---|---|
| methyl parabene PH Eur | 0.61 g |
| polyparabene PH Eur | 0.33 g |
| sodium sorbate PH Eur | 1.27 g |
| purified water PH Eur | 993.48 g |
| lemon aroma | 1.00 g |
| peppermint oil | 0.20 g |
| citric acid | 2.00 g |
| sodium selenite pentahydrate | 1.11 g |
| aerosil 200 | 30.00 g |
| | 1030.00 g |

A dose of 3 to 5 drops of this selenite solution containing silicate was applied to the affected skin parts 5 times a day. In all 3 patients the papilloma warts disappeared within a period of 3 weeks. Treatment was stopped after the warts had disappeared.

1. The use of preparations containing selenite or selenate, mixed with a pharmaceutically acceptable or food compatible acid, with the exception of ascorbic acid, for the preparation of an agent for the treatment of wounds.

2. The use according to claim 1, characterized in that said agent is used for the treatment of necrotic processes in wounds.

3. The use according to claim 2 for the preparation of an agent for the treatment of wounds in the dental region, preferably for the treatment of tooth extraction wounds, particularly for the treatment of dolor post extractionem or for the treatment of parodontal conditions.

4. The use of preparations containing selenite or selenate, mixed with a pharmaceutically acceptable or food compatible acid, with the exception of ascorbic acid, for the preparation of an agent for the treatment of microcirculatory disorders.

5. The use of preparations containing selenite or selenate, mixed with a pharmaceutically acceptable or food compatible acid, with the exception of ascorbic acid, for the preparation of an agent for the treatment of local hypoxemia.

6. The use of preparations containing selenite or selenate, mixed with a pharmaceutically acceptable or food compatible acid, with the exception of ascorbic acid, for the preparation of an agent for the treatment of cellular damage due to lipid peroxidation.

7. The use according to any of claims 1 to 6, characterized in that said wounds are mechanical wounds, particularly wounds originating from medical and surgical interventions, respectively.

8. An agent for the treatment of wounds, characterized in that it comprises a preparation containing selenite or selenate mixed with a pharmaceutically acceptable or food compatible acid, with the exception of ascorbic acid, and a pharmaceutically acceptable carrier.

9. An agent for the treatment of wounds according to claim 8, characterized in that said preparation containing selenium is provided on a solid surface, particularly as a plaster or wound implant.

10. An agent for the treatment of wounds according to claim 8, characterized in that it is provided in the form of a gel.

11. Toothpaste, characterized in that it comprises a preparation containing selenite or selenate mixed with a pharmaceutically acceptable or food compatible acid, with the exception of ascorbic acid, and a pharmaceutically acceptable carrier.

12. A mouth wash, characterized in that it comprises a preparation containing selenite or selenate mixed with a pharmaceutically acceptable or food compatible acid, with the exception of ascorbic acid, and a pharmaceutically acceptable carrier.

13. An agent according to any one of claims 8 to 12, characterized in that said acid is selected from citric acid, malic acid, carbonic acid, acetic acid, various fruit acids or mixtures thereof.

14. An agent according to any one of claims 8 to 13, characterized in that it is prepared for buccal or topical administration.
